(19) United States
(12) Patent Application Publication
(10) Pub. No.: US 2015/0012086 A1
(43) Pub. Date: Jan. 8, 2015

(54) FUNCTIONAL SIZER FOR A HEART VALVE IMPLANTABLE DEVICE

(71) Applicant: Levi BASSIN, (US)

(72) Inventor: Levi Bassin, Bellevue Hill (AU)

(21) Appl. No.: 14/376,342

(22) PCT Filed: Mar. 8, 2013
(86) PCT No.: PCT/AU2013/000241

§ 371 Date: Aug. 1, 2014

(30) Foreign Application Priority Data
Mar. 9, 2012 (AU) 2012900931

(51) Int. Cl. A61F 2/24

(52) U.S. Cl. CPC A61F 2/2445; A61F 2/2427; A61F 2/2472; A61F 2220/0075
USPC 623/2.17

(57) ABSTRACT

A functional sizer and method of sizing that overcome the problems of relying purely on visual assessment of size in selecting an implantable heart valve device (repair implant or a heart valve replacement prosthesis) and of being unable to test the fit and performance of the heart valve implantable device until after it has been stitched in position. The invention provides a functional sizer that is functional and can be reversibly secured in position, largely using the valve sutures under tension, thereby enabling assessment of fit and performance prior to permanently stitching a heart valve prosthesis.

### TECHNICAL FIELD

The present invention relates to methods and devices for heart valve repair and replacement, and in particular to sizer devices for sizing an implantable device for heart valve repair and/or replacement.

### COPYRIGHT NOTICE

This document is subject to copyright. The reproduction, communication and distribution of this document is not permitted without prior consent from the copyright owner, other than as permitted under section 226 of the Patents Act 1990.

### BACKGROUND

There are four one-way valves in the heart to enable flow of blood in one direction. These are:

1. the two atrioventricular valves between the atria and ventricles—namely:
a. the mitral valve; and
b. the tricuspid valve; and

2. the semilunar valves, in the arteries leaving the heart—namely:
a. the aortic valve; and
b. the pulmonary valve.

Heart valve disease can occur in which one or more of the heart valves fails to function properly. This may involve the valve becoming incompetent or not closing tight enough, allowing “regurgitation” or “leaking” of blood in a retrograde direction.

Various surgical techniques are used to replace or repair a “leaky” valve. Repair is preferable to replacement if at all possible. Taking atrioventricular valve (mitral or tricuspid valve) repair as an example, the preferred surgical approach for repair involves, in part, implanting an annuloplasty ring or band. This is a strong artificial complete ring or partial ring (band) secured to the valve annulus, and which pulls together valve leaflets making up the valve. This functions to remodel the “annulus” of the valve (a circular ring of flexible, fibrous and/or muscular tissue that surrounds the opening of the valve and to which the leaflets attach) smaller or larger and/or to prevent further dilatation of the opening, thereby restoring the physiological form and function of the normal valve apparatus.

There are many annuloplasty bands or rings in different shapes and sizes commercially available. This is to account for a variety of valve sizes and functional requirements. In order to determine which size of annuloplasty ring to use, a surgeon will often use a “sizer”, or ring template as a guide. The surgeon estimates the size of the valve annulus by inserting a sizer adjacent to and in alignment with the valve annulus and then visualising whether the annulus is bigger or smaller than the sizer (template). This step may be repeated until a sizer is identified that most closely corresponds to the size of the valve annulus that the surgeon determines is appropriate for the given pathology. The surgeon then selects a correspondingly sized annuloplasty ring. Sometimes the surgeon aims for the same sized annulus as the template, and sometimes smaller or larger. For example, frequently, a surgeon will “downsize” by one or two levels, selecting a ring one or two sizes down from the size of the sizer.

Other examples of heart valve surgery involving an implantable device implanted into a heart valve, and in which correctly sizing the implantable device is important, include:
(a) aortic valve repair as a result of an aortic aneurysm. A surgeon will replace a diseased aorta with an artificial tube (graft) and reattach the aortic valve to the tube;
(b) pulmonary valve repair in which an artificial tube is attached to a diseased pulmonary artery;
(c) heart valve repairs using artificial mitral or tricuspid chords (neo-chords);
(d) heart valve replacement surgery, in which an artificial valve (valve replacement) is implanted to replace a diseased valve.

In both aortic and pulmonary valve repair, the size of the tube graft is critical in re-establishing correct function of the valve. Currently surgeons approximate the size using a template similar to the process used in mitral valve surgery. However as with mitral valve repair, the surgeon cannot accurately test the efficacy of the repair until the tube graft is permanently sutured to the heart.

In heart valve repairs using artificial atrioventricular (i.e. mitral or tricuspid) chords (neo-chords), chord length is altered when an annuloplasty ring is positioned in place. Currently it is not possible to assess the effect of an annuloplasty ring on chord length until the annuloplasty ring has been sutured in place. This poses a difficulty for surgeons attempting chordal replacement.

In valve replacement surgery, surgeons currently use a template and hold it in position for visually sizing the valve and its replacement. However, a valve replacement alters the architecture of the heart and can therefore change the size of an adjacent valve annulus. The, impact is not known until permanent fixing of the valve replacement. This is particularly problematic when implanting two adjacent artificial valves at the same time.

As described above, the sizing of implantable heart valve devices (repair devices and replacement prostheses), although critical to the performance of heart valve repair and replacement, still relies on relatively crude methods of sizing that are often unreliable. Sizer devices such as those described in US 2002/0133180 and US 2009/0192605 have been developed that include markings to delineate a distance between selected points of a valve annulus or other measurement. WO 2010/090720 describes a sizer that provides dimensional information by allowing a surgeon to view the annulus through the sizer and assessing the anatomic position of the valve annulus beneath the sizer relative to “ribs” positioned on the sizer. This provides a gauge of the dimensions of the valve annulus. These sizer devices remove the reliance on visual approximation alone. However, they suffer a common disadvantage in that the sizer device only provides size information without providing any means to test function.

The difficulty is that the mitral valve annulus changes in shape and size during the cardiac cycle. Accordingly, it is difficult to accurately size an annuloplasty ring based on measurements or visual assessment of size alone. Ultimately, correct fit is indicated by a competent valve, as assessed during surgery by testing for leakage. For example, even with an accurately measured annuloplasty ring, the surgeon can only test the efficacy of, the mitral valve repair after inserting the selected annuloplasty ring, stitching the ring into position, assessing the effect of the annuloplasty ring on chord length if the repair will also involve using neochords, and then injecting saline into the ventricular cavity causing closure of the atrioventricular valve. Through this process is the surgeon able to determine if the mitral valve is working properly (i.e. has been repaired or replaced successfully). Similar principles and difficulties apply for sizing and testing correct fit (functionality) of tube grafts for aortic or pulmonary valve repair or replacement.

Once the implantable device for heart valve repair and/or replacement (e.g. annuloplasty ring, tube graft or valve replacement) is secured in place, there are risks to the patient to remove an ill-fitting prosthesis and re-insert a better fitting one. This includes risks arising from:
(a) the additional time required to remove and replace the prosthesis. This is specifically because the patient is connected to a heart-lung machine for the duration of the surgery. The longer the patient remains connected to the heart-lung machine, the more deleterious the effects and the higher the risk of stroke, heart damage, and bleeding in particular. The risk is proportional to the duration of time spent the patient is connected to the heart-lung machine;
(b) damage to the heart from removing and restitching the implant, all the above taking place while the heart is cooled and stopped.

The risks are sufficiently significant that, for example in mitral valve repair, many surgeons would leave a less than perfect result rather than risk removal and replacement of the annuloplasty ring. An additional disadvantage is that each implantable heart valve device (e.g. annuloplasty ring, graft or neochord) is very costly and only usable once (as it cannot be sterilised and the insertion of stitches damages the structure). Therefore, stitching in an implantable heart valve device (e.g. annuloplasty ring, graft or neochord) means that if it needs to be removed due to ill-fit, it must then be discarded. This makes re-sizing of an implantable heart valve device by replacement a very expensive process.

There is a need for a functional sizer that would allow a surgeon to accurately assess, fit and test function of an implantable heart valve device for heart repair or replacement surgery without the need to stitch in a working implantable device, and run the risk of ill-fit, or the need to replace the implantable device (with attendant risks of damage to stitches and risk to patient).

It is an object of the present invention to provide a functional sizer that allows a surgeon to accurately assess fit and test function of an implantable heart valve device during heart repair or replacement surgery without the need to permanently fix an implantable heart valve device.

### SUMMARY

According to an aspect of the invention there is provided a functional sizer for sizing an implantable heart valve device for heart valve repair or replacement, the functional sizer comprising a ring-like body with a central hole, the ring-like body being of a sterilisable material,

wherein the ring-like body is one of the following two-dimensional shapes:
(a) a closed loop;
(b) a penannular shape, and

wherein the ring-like body substantially corresponds in inner circumference to a ring of suture threads positioned around a heart valve annulus for securing a heart valve implantable device of a predetermined size, each suture thread of the ring of suture threads having a free end

wherein the functional sizer is adapted to be reversibly secured in position against the heart valve annulus, the reversibly secured functional sizer simulating an effect of a heart valve implantable device of a substantially corresponding size and form, thereby facilitating assessment of fit and function of the heart valve implantable device.

According to another aspect of the invention there is provided a functional sizer for sizing an implantable heart valve device for heart valve repair or replacement, the functional sizer comprising a ring-like body with a central hole, the ring-like body being of a sterilisable material,

wherein the ring-like body is one of the following two-dimensional shapes:
(a) a closed loop;
(b) a penannular shape, and

wherein the ring-like body substantially corresponds in inner circumference to a ring of suture threads positioned around a heart valve annulus for securing a heart valve implantable device of a predetermined size, each suture thread of the ring of suture threads having a free end

wherein the functional sizer'is adapted to be reversibly secured in position against the heart valve annulus, the reversibly secured functional sizer simulating an effect of a heart valve implantable device of a substantially corresponding size and form, thereby facilitating assessment of fit and function of the heart valve implantable device, and wherein said functional sizer is further adapted to be permanently secured in position on a heart annulus by one of the following:
(a) stitching to the heart annulus;
(b) retaining the self-clamping mechanism in a closed position;
(c) retaining the magnetised cap on the ring-like body of the functional sizer.

According to a further aspect of the invention there is provided a method of sizing a heart valve implantable device including the steps of:
(a) positioning a functional sizer in a heart, wherein the functional sizer is a ring-like body of sterilisable material of an approximately corresponding size to a heart valve implantable device of a predetermined size;
(b) reversibly securing the functional sizer in position.

According to another further aspect of the invention there is provided a method of sizing a heart valve implantable device including the steps of:
(a) positioning a functional sizer against a heart valve annulus, wherein the functional sizer comprises a ring-like body with a central hole, the ring-like body being of a sterilisable material, wherein the ring-like body is one of the following two-dimensional shapes:
   i. a closed loop;
   ii. a penannular shape, and wherein the ring-like body substantially corresponds in inner circumference to a ring of suture threads positioned around the heart valve annulus for securing a heart valve implantable device of a predetermined size, each suture thread of the ring of suture threads having a free end;
(b) drawing the free ends of said suture threads up through the ring-like body from underneath to over the ring-like body;
(c) placing the one or more suture threads under tension after drawing the free ends up through and over the ring-like body such that when reversibly secured in position against the heart valve annulus by placing the one or more suture threads under tension, the functional sizer simulates an effect of a heart valve implantable device of a substantially corresponding size, shape and form, thereby facilitating assessment of fit and function of the heart valve implantable device.

### DETAILED DESCRIPTION

The invention thus provides a functional sizer and method of sizing that overcome the problems of relying purely on visual assessment of size in selecting an implantable heart valve device (repair implant or a heart valve replacement prosthesis) and of being unable to test the fit and performance of the heart valve implantable device until after it has been stitched in position. The invention provides a functional sizer that is functional and can be reversibly secured in position, largely using the valve sutures under tension, thereby enabling assessment of fit and performance prior to permanently stitching a heart valve prosthesis.

For a better understanding of the invention and to show how it may be performed, a preferred embodiment will now be described, by way of non-limiting example only, with reference to the accompanying drawings and examples.

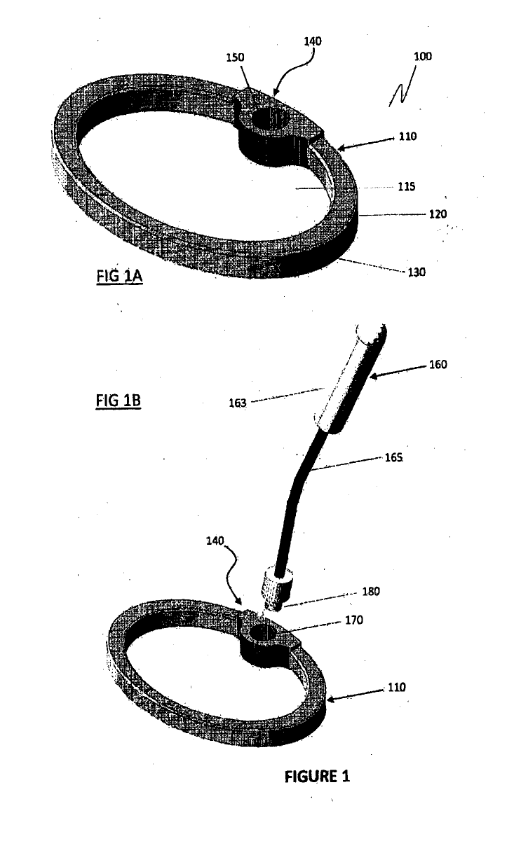

FIG. 1A is a perspective view of a functional sizer according to an embodiment of the invention.

FIG. 1B shows the functional sizer of FIG. 1A and an applicator for handling the functional sizer according to an embodiment of the invention.

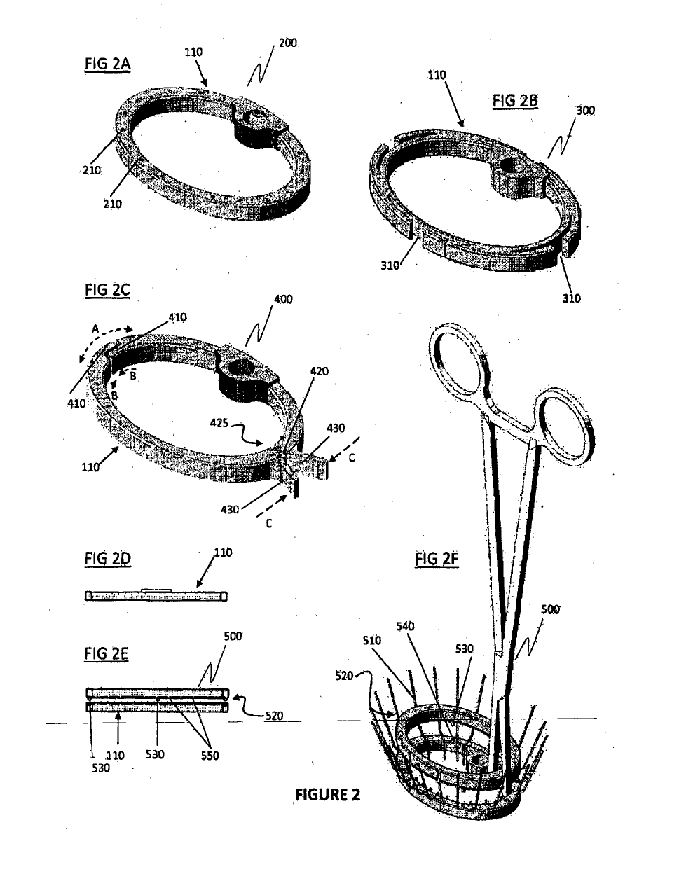

FIG. 2 shows different embodiments of a functional sizer having securing means.

FIG. 2A is a perspective view of a functional sizer with preformed suture holes as the securing means.

FIG. 2B is a perspective view of a functional sizer with preformed suture slots as the securing means.

FIG. 2C is a perspective view of a functional sizer with a self-clamp mechanism as the securing means.

FIGS. 2D and 2E show side views of an embodiment that has a magnetic cap as the securing means.

FIG. 2F is a perspective view of the embodiment of FIG. 2E showing the positioning of sutures relative to the magnetic cap.

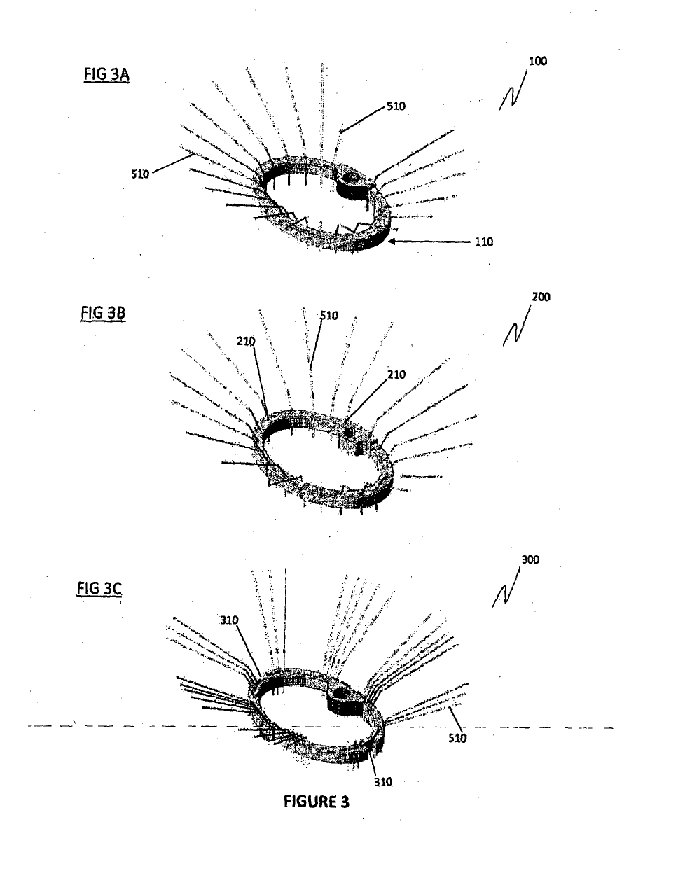

FIGS. 3A to 3C contain perspective views of the functional sizer of FIGS. 1A, 2A and 2B, respectively, showing the free ends of suture threads positioned in a heart valve annulus extending up and over (FIG. 3A) the functional sizer or fed through preformed suture holes (FIGS. 3B) or suture slots (FIG. 3C) in the functional sizer.

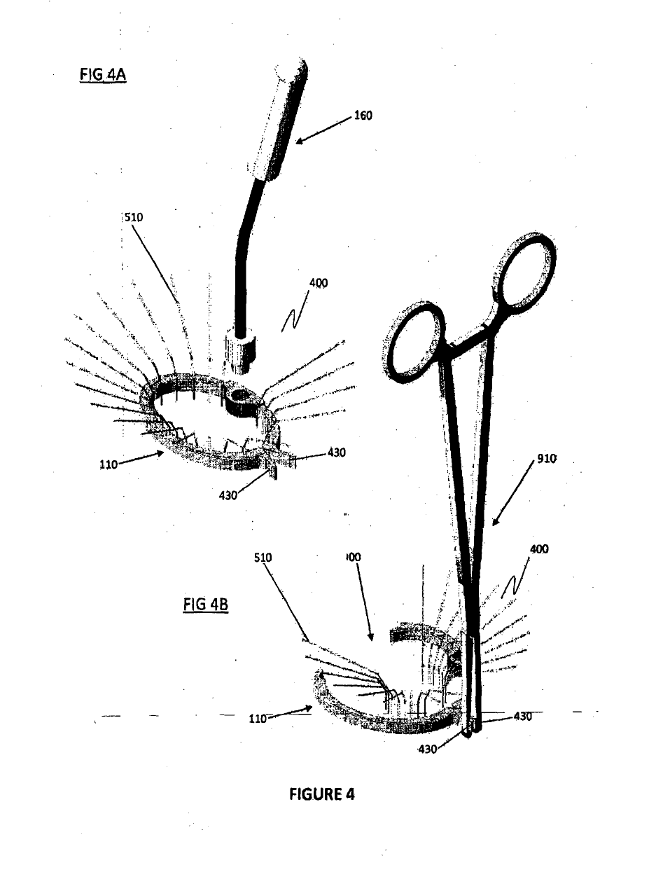

FIG. 4 contains perspective views of the functional sizer of FIG. 2C in a closed position (FIG. 4A) and held in an open position by a conventional pair of forceps (FIG. 4B).

FIG. 5 is a flowchart of a method for sizing a heart valve annulus according to an embodiment of the invention.

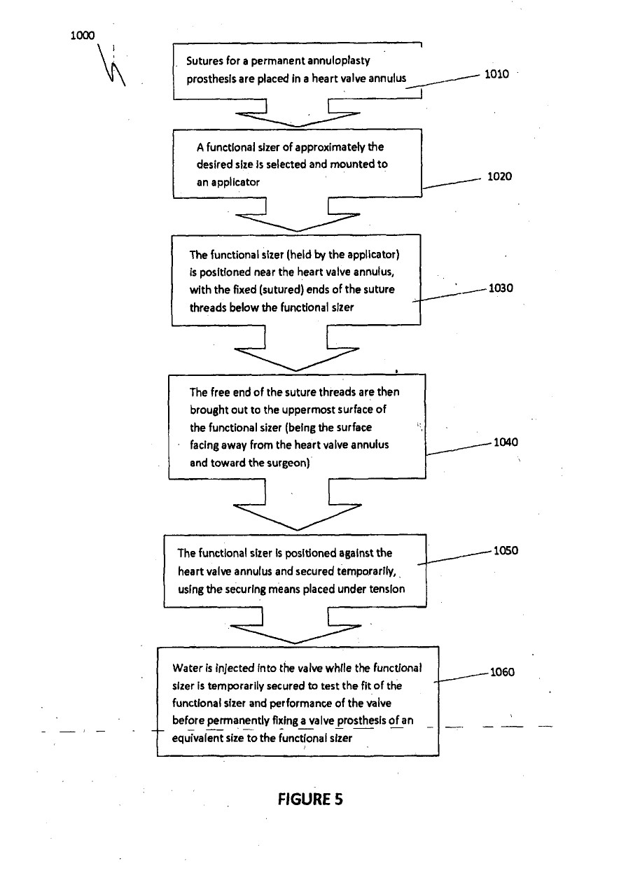

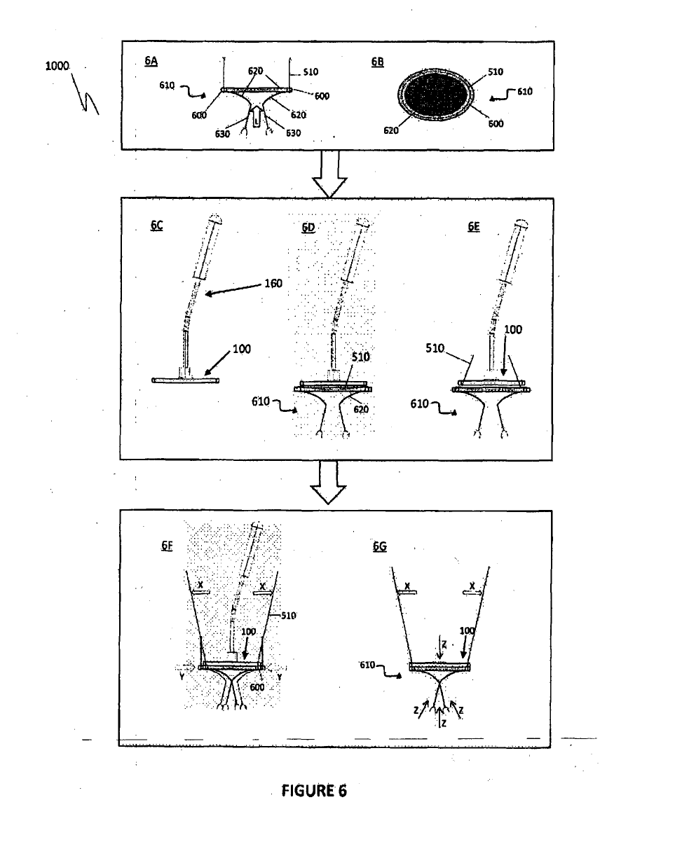

FIG. 6 is a schematic diagram of the method of FIG. 5 for sizing a valve annulus and implantable heart valve device. Shown by way of example is the method of Example 1: mitral valve repair, in which the implantable device is a repair implant.

FIG. 6A is side view of a mitral valve in cross section.

FIG. 6B is a plan view of a mitral valve showing a ring of sutures in the annulus.

FIG. 6C is a side view of a functional sizer mounted to an applicator.

FIG. 6D is a side view of a mitral valve in cross section, showing the functional sizer positioned against the annulus with the sutures underneath the sizer.

FIG. 6E shows the functional sizer of FIG. 6D with the sutures drawn up through the centre of the sizer.

FIG. 6F shows the functional sizer of FIG. 6E secured in position with tension (applied to the sutures in the direction shown), reshaping the heart valve annulus and drawing the leaflets together.

FIG. 6G shows the direction of saline injected into the valve while the functional sizer is secured, to test the performance of the valve before fixing a permanent annuloplasty ring.

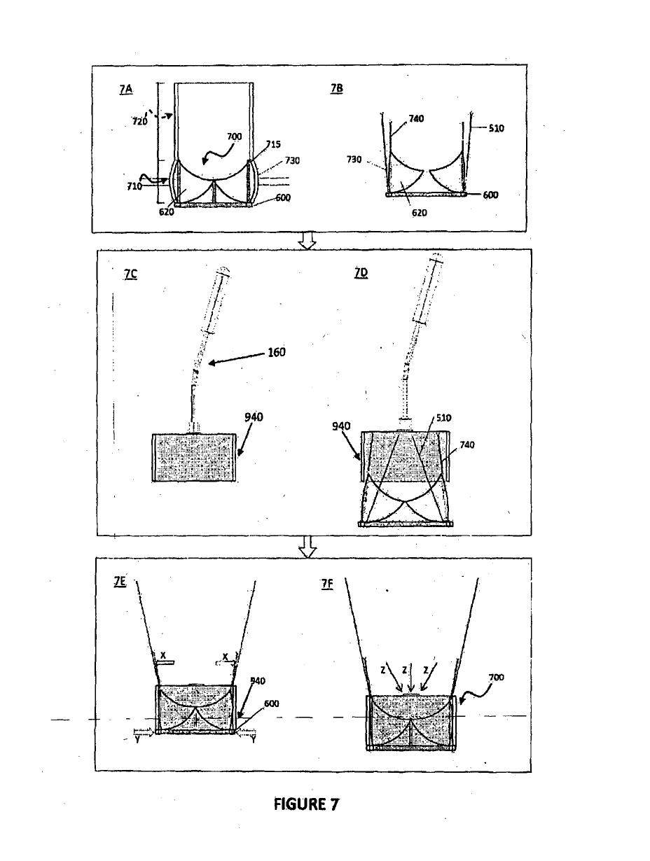

FIG. 7 is a schematic diagram of the method of FIG. 5 for sizing a valve annulus and implantable heart valve device. Shown by way of example is the method of Example 2: aortic valve repair, in which the implantable device is an aortic graft.

FIG. 7A is a side view of an aortic valve in cross section.

FIG. 7B is a side view of an aortic valve showing sutures in position in the commissures and in the annulus.

FIG. 7C is a side view of a functional sizer mounted to an applicator.

FIG. 7D is a side view of an aortic valve in cross section, showing the functional sizer positioned above the annulus with the sutures being drawn up through the centre of the sizer, from underneath.

FIG. 7E is a side view of an aortic valve in cross section, showing the functional sizer positioned against the annulus with the sutures placed under tension.

FIG. 7F shows the direction of saline injected into the valve while the functional sizer is secured, to test the performance of the valve before fixing a permanent prosthesis.

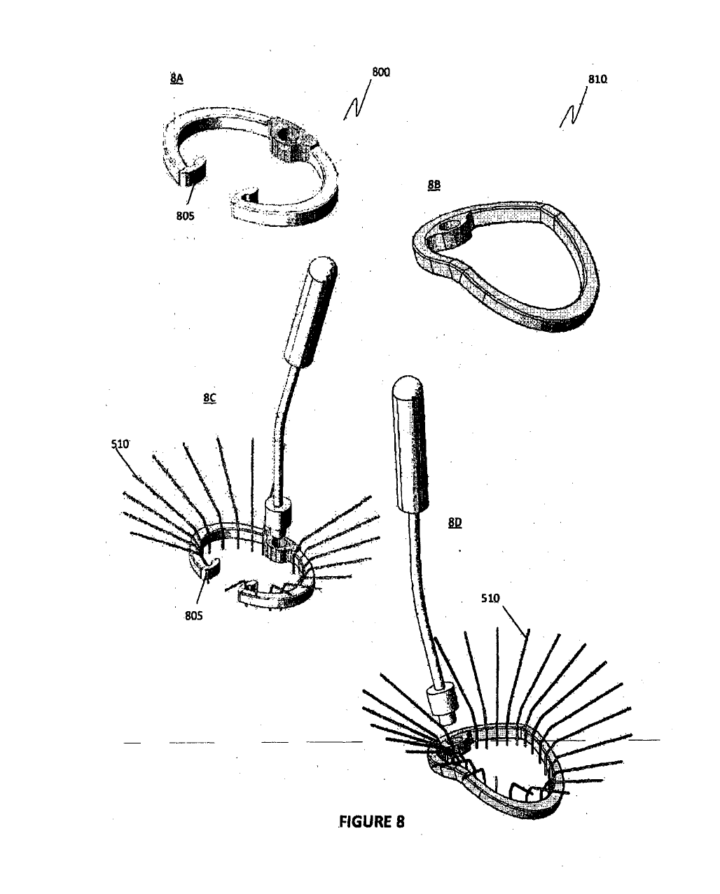

FIG. 8 shows alternative embodiments to those shown in FIGS. 1 to 4, being functional sizers suitable for use in mitral or tricuspid valve repair.

FIG. 8A is an open loop embodiment (partial ring/band) of a functional sizer.

FIG. 8B is a three-dimensional “bent” ring embodiment of a functional sizer.

FIGS. 8C and 8D show the embodiments of FIG. 8A and 8B, respectively, with the free ends of suture threads fixed in a heart annulus being brought up and over the functional sizer for securing by tension.

FIG. 9 shows exemplary embodiments of a functional sizer suitable for use in aortic or pulmonary valve repair.

FIG. 9A is a perspective view of a ring-like embodiment.

FIG. 9B is a perspective view of a ring-like embodiment with preformed suture slots.

FIG. 9C is a perspective view of a tube-like embodiment.

FIG. 9D is a perspective view of a ring-like embodiment with a self-clamping mechanism.

FIG. 9E is a perspective view of the functional sizer of FIG. 9A showing the free ends of sutures fixed in a heart annulus travelling up and over the sizer ring. Also shown schematically are sutures in the commissures sitting against the inner circumference of the functional sizer ring then up and over the sizer ring with the other sutures.

FIG. 9F is a perspective view of the functional sizer of FIG. 9B showing the free ends of sutures in the heart annulus fed through the slots of the ring. The sutures from the commissures are not fed through the slots but sit against the inner circumference of the functional sizer ring.

FIG. 9G is a perspective view of a tube-like embodiment with suture slots showing sutures fed through the slots and the position of commissure sutures within the tube.

FIG. 9H is the embodiment of FIG. 9C showing the free ends of sutures fixed in a heart annulus travelling up and over the sizer tube, through the centre of the tube. Also shown is the position of commissure sutures within the tube.

FIG. 9I is a perspective view of a bulbous embodiment having a ring with a self-clamping mechanism and a bulb-like structure to house the commissures.

FIG. 9J is a perspective view of the embodiment of FIG. 9D in a closed position. Also shown is an applicator and sutures (both from the annulus and commissures) in position.

FIG. 9K is a perspective view of the embodiment of FIG. 9J in an open position.

FIG. 9L is a perspective view of a tubular embodiment with a self-clamping mechanism. The functional sizer is in a closed position, the sutures from the annulus and commissures in position.

FIG. 9M is the embodiment of FIG. 9L in an open position.

FIG. 9N is a section view of an aortic valve showing the shape of the graft which would require a functional sizer for use in sinotubular junction dilatation repair.

FIG. 9O is a section view of an aortic valve showing the shape of the graft which would require a functional sizer for use in an annuloaortic ectasia repair.

FIG. 9P is a section view of an aortic valve showing the shape of the graft which would require a functional sizer for use in an aortic root graft in an aortic root aneurysm repair.

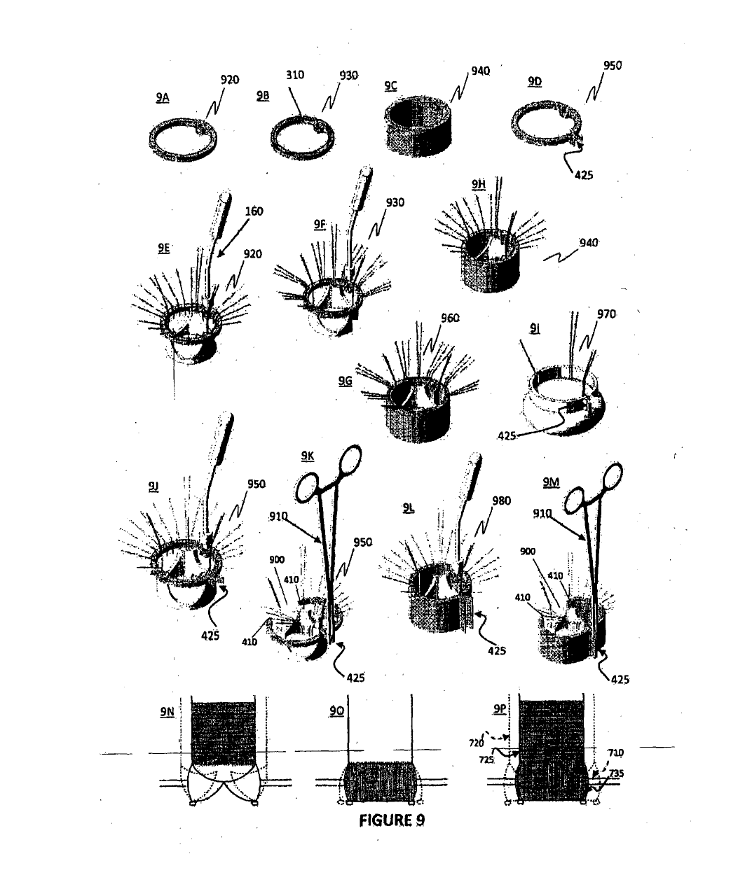

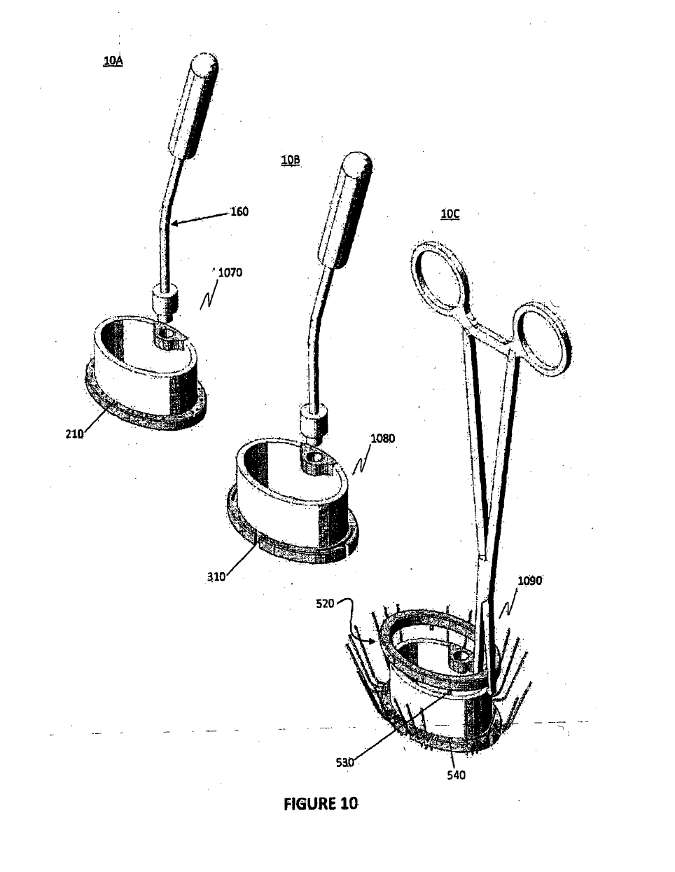

FIG. 10 shows perspective views of a functional sizer for a heart valve replacement implantable device according to various embodiments of the invention.

FIG. 10A is an embodiment including preformed suture holes as securing means.

FIG. 10B is an embodiment including preformed suture slots as securing means.

FIG. 10C is an embodiment including a magnetic cap as the securing means.

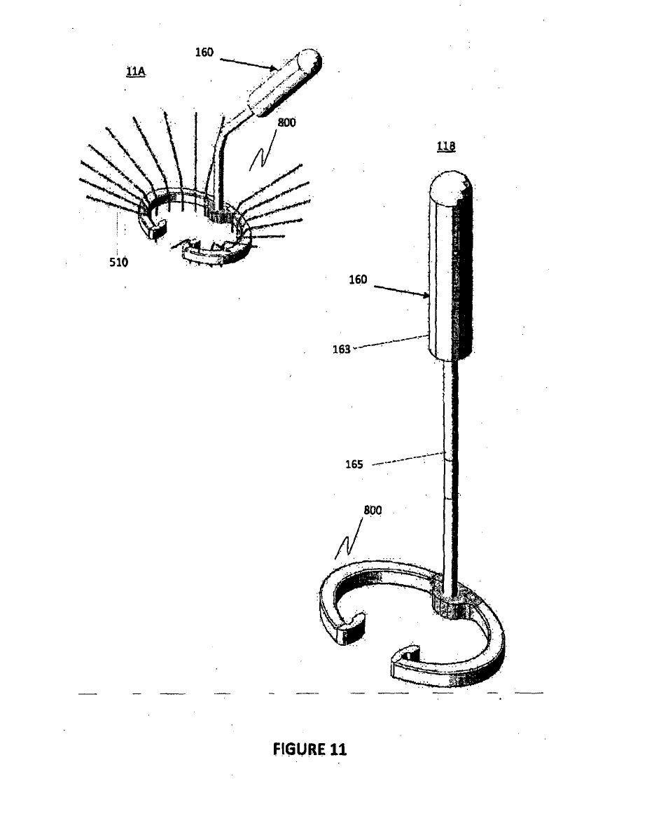

FIG. 11 shows perspective views of the functional sizer of FIG. 8A with a permanently fixed applicator.

FIG. 11A shows the functional sizer with sutures in position. The shaft of the applicator is angled.

FIG. 11B shows the functional sizer of FIG. 8A without sutures. The shaft of the applicator is straight.

### EXAMPLE 1

### A Method for Sizing an Annuloplasty Ring for Mitral Valve Repair

### EXAMPLE 2

### A Method for Sizing a Supracoronary Graft for Aortic Valve Repair

The elements of the invention are now described.

### DETAILED DESCRIPTION OF PREFERRED EMBODIMENTS

The invention provides a functional sizer for sizing a heart valve annulus and an implantable device for heart valve repair and/or heart valve replacement.

Referring to FIG. 1A, in an embodiment the functional sizer 100 comprises a ring-like body 110 with a central hole 115, the ring-like body 110 having an inner circumference 120 and an outer circumference 130. The ring-like body 110 is made from a sterilisable material to allow removal and re-use.

The specific two-dimensional shape and three-dimensional form of the body 110 may vary (as required for a specific repair) and can be any suitable 2D shape or 3D form in current or future production, including any:
(a) closed loop (e.g. the substantially planar circular or oval 2D shapes of FIGS. 1 to 4, or the three-dimensional forms illustrated in FIGS. 8B, 9 and 10, including tube-like forms, and a “bent” ring form or a bulbous ring form as examples of a 3D ring-like form); or
(b) a penannular (i.e. partial or incomplete ring/band) shape (e.g. see FIG. 8A). Again, the penannular ring-like bodies may be substantially planar (2D) shapes (circular, oval) or of a 3D form (e.g. tube-like as shown in FIGS. 9G, 9H, 10A, 10B), bulbous (FIG. 9I) or bent-ring (FIG. 8B).

Persons skilled in the art would appreciate that shapes other than the specific shapes illustrated may also be suitable for repair or replacement of a heart valve.

The inner circumference 120 of the body 110 (see FIG. 1A) substantially corresponds in size to an annuloplasty ring of a predetermined size (circumference) for any type of heart valve repair or replacement. The inner circumference 120 of the body 110 corresponds to the placement of a line of sutures along the central circumference (midway between inner and outer diameter) of the correspondingly-sized annuloplasty ring. Various sizes of body 110 are provided corresponding to various predetermined sizes of annuloplasty ring.

The ring-like body 110 includes a fixture mechanism 140 as shown in FIG. 1A. The fixture mechanism 140 includes a mounting means 150 for mounting the body 110 to an applicator (item 160 in FIG. 1B). Mounting the body 110 to an applicator 160 enables the functional sizer 100 to be handled by handling the applicator 160.

An embodiment of the applicator 160 is shown in FIG. 1B and includes a shaft 165 having:
(a) a handle means 163 at one end; and
(b) a threaded mount 180 at the far end.

The mounting means (labelled 150 in FIG. 1A) of the functional sizer is any suitable means, for example a thread (labelled 170 in FIG. 1B) within the mounting means 150 that corresponds to a thread on the threaded end 180 of applicator 160. This allows the applicator 160 to be screwed into the mounting means 150 of the functional sizer for application of the functional sizer to the heart or otherwise handling the functional sizer. This reversible form of mounting means also allows the removal of the applicator 160 from the functional sizer.

In an alternative embodiment, the applicator 160 is fixed rather than removable from the functional sizer (e.g. see FIGS. 11A and 11B). In this embodiment, the mounting means is a permanent form of securing an applicator to a functional size (e.g. a mechanical connection [nuts, bolts, fasteners], adhesive, welding). FIG. 11A shows the open loop functional sizer (partial ring/band) 800 of FIG. 8A including a permanently fixed applicator 160. This embodiment is shown by way of example only. Any of the other exemplary embodiments of the functional sizer may also include a fixed applicator. FIG. 11B shows the applicator 160 in position. FIG. 11A shows annular sutures 510 placed in position within the functional sizer 800.

The shaft 165 of the applicator 160 extends between the ring-like body of the functional sizer to a handle means and can be produced in a variety of lengths depending on the requirement (e.g. from 10 to 30 cm). The applicator is reusable and produced from any suitable sterilisable material, including a rigid material or a malleable material (e.g. a malleable metal) so that it can be bent and shaped for every case and to provide access as required. There is a grip forming a handle 164 at the end of the shaft 165, for holding. The entire applicator 160 is reusable and sterilisable. In the embodiment depicted in FIG. 11, the applicator 160 is permanently fixed to the functional sizer. The complete unit (functional sizer with applicator) is reusable and sterilisable. In FIG. 11A the shaft is angled and in FIG. 11B the shaft of the applicator 160 is straight.

Referring to FIG. 6D, the functional sizer 100 is positioned near a heart valve annulus 600. The annulus 600 has a ring of sutures 510 previously placed around the heart valve annulus 600 by the surgeon. The sutures 510 sit below the functional sizer 100 after the sizer 100 is placed in position (see FIG. 6D). The free ends of the suture threads 510 are drawn up through the centre of the functional sizer 100 and over the body 110 then tension is applied in the direction of the free ends of the sutures 510 (see FIGS. 6E and 6F). Applying tension secures the functional sizer 100 in position.

In other embodiments, securing means (e.g. as shown in FIGS. 2, 3, 8, 9 and 10) are used to hold the functional sizer in position for testing. These may include a self-clamping mechanism (e.g. item 425 in FIG. 2C described below) or a magnetised cap (item 520 in FIG. 2F described below).

The invention also provides a method for sizing a heart valve annulus and an implantable device suitable for repair or replacement of the heart valve. In a preferred embodiment, the method 1000 (FIG. 5) includes the following steps:
1. sutures (refer item 510 in FIGS. 6A and 6B) for a final permanent annuloplasty ring or valve replacement are placed in a heart valve annulus 600 (step 1010 in FIG. 5);
2. a functional sizer of approximately the desired size is selected and mounted to an applicator 160 (step 1020, FIG. 6C). Alternatively, the functional sizer is already permanently mounted to an applicator. The functional sizer can be “upsized” or “downsized” if required, according to a surgeon's preference for a specific surgery. Embodiments 200 (FIG. 2A), 300 (FIG. 2B), 930 (FIG. 9B) and 960 (FIG. 9G) are suitable for upsizing;
3. the functional sizer 100 (held by the applicator 160) is positioned near the heart valve annulus, with the fixed (sutured) ends of the threads 510 below the functional sizer 100 (step 1030, FIG. 6D);
4. the free ends of the suture threads 510 are then brought out to the uppermost surface of the functional sizer 100 (the uppermost surface being the surface facing away from the heart valve annulus and toward the surgeon)—step 1040 and FIG. 6E;
5. the functional sizer is positioned against the heart valve annulus and the threads are placed under tension (step 1050). This is achieved by attaching the threads 510 to a securing device of some sort—for example, a valve suture guide or ratcheted forceps that clamp the threads 510 and is then placed on the patient's chest. FIG. 6F shows the direction of force (tension) applied to the sutures 510 (arrows X) and corresponding effect on the heart valve annulus (reshaping or resizing of the annulus in the direction indicated by arrows Y);
6. the performance of the heart valve 610 is tested with the functional sizer 100 held in position by tension on sutures 510 (see FIG. 6G). This is done by injecting saline in the directions indicated by arrows Z.

By placing the suture threads 510 under tension, force is applied to the functional sizer to secure the functional sizer 100 in position on the heart valve annulus. The force applied to the functional sizer 100 also causes the annulus to conform to the same shape as the functional sizer (which reflects the size and shape of a permanent heart valve implantable device (e.g. annuloplasty ring, tubular graft or artificial valve). In this way, the secured functional sizer reshapes the underlying heart valve annulus to the shape of the functional sizer 100, thereby simulating the effect of an annuloplasty ring of a corresponding size of the selected functional sizer 100. The functional sizer 100 can be used to temporarily reshape:
(a) the annulus of any heart valve for valve replacement;
(b) the aorta or pulmonary artery at the top of the commissures for repair;
(c) the mitral or tricuspid valve for repair.

The secured functional sizer 100 can then be tested using conventional means for assessing fit and functionality (prevention of leakage). Specific examples are provided below.

As the mitral valve has a similar structure to the tricuspid valve, and the aortic valve is similar in structure to the pulmonary valve, the examples below are limited one of each similar structure.

### EXAMPLE 1

### A Method for Sizing an Annuloplasty Ring for Mitral Valve Repair

This example illustrates use of a functional sizer for sizing an implantable heart valve device in the form of an annuloplasty ring.

FIG. 6A is a side view of a mitral valve 610 in cross section. The annulus 600 travels circumferentially around the mitral valve leaflets 620. This can be better seen in plan view in FIG. 6B. In a healthy valve (not shown), the leaflets are closed so that no leakage can occur between the leaflets. A chordal structure (mitral chords or neochords—labelled 630) prevent prolapse of the valve leaflets. In a diseased valve, the annulus is dilated and the valve leaflets do not close tightly, allowing blood to leak or flow, “backwards”—that is, into the atria. FIG. 6B shows a dilated annulus and the leaflets 620 apart, allowing blood to leak back into the atria (in the direction indicated by arrow L). Replacement of the chords with artificial chords may be part of a mitral valve repair.

Sutures for a permanent annuloplasty ring are placed around a mitral valve annulus. The sutures 510 are indicated by the dotted line in the annulus 600 of the mitral valve 610 depicted in FIG. 6B. The free ends of the suture threads 510 extend away from the annulus, as can be seen schematically in FIG. 6A.

A functional sizer 100 of approximately the size of the heart valve annulus is selected and mounted to an applicator 160 (FIG. 6C). Alternatively, a functional sizer of the form shown in FIG. 9C but with a permanently fixed applicator (such as depicted in FIG. 11) is selected. The functional sizer 100 is positioned near the annulus with the sutures 510 underneath the functional sizer 100, between the functional sizer 100 and the valve leaflets 620 (FIG. 6D). The sutures 510 are drawn up through the centre of the functional sizer 100 (FIG. 6E). Tension is applied to the sutures 510 in the direction indicated by arrows X (FIG. 6F), to secure the functional sizer 100 in position on the annulus 600. This is achieved by attaching the threads 510 to a securing device of some sort—for example, a valve suture guide or ratcheted forceps that clamp the threads 510 and is then placed, on the patient's chest.

In this way, the heart valve annulus is reshaped or conforms to the shape of the functional sizer—the annulus is no longer dilated and the leaflets are drawn together, to close the valve.

The performance of the valve 610 is then tested (FIG. 6G) while the functional sizer 100 is temporarily secured to the annulus (here, by placing the sutures in the annulus under tension). Saline is injected in the directions indicated by arrows Z. If there is no leakage, this indicates a good “fit” of the functional sizer 100 to the annulus 600 and that the valve is closed (functioning correctly).

As the method of securing the functional sizer 100 in position is readily reversible, the functional sizer 100 can be removed without damage to the heart valve annulus. An annuloplasty ring that corresponds in size to the functional sizer can then be fixed permanently, having previously gauged the appropriate size using the functional sizer.

Alternatively, if there is saline leakage during performance testing, indicating a poor fit, the functional sizer can be removed and a new functional sizer of a different size tested. The removal and replacement process is much faster than the time required for removing and replacing a permanently stitched but ill-fitting implant.

### EXAMPLE 2

### A method for Sizing an Aortic Graft for Aortic Valve Repair

This example illustrates use of a functional sizer for sizing an implantable heart valve device in the form of an aortic graft.

The functional sizer can be used to temporarily remodel the aortic annulus/root/sinotubular junction (STJ) to simulate an aortic graft. FIG. 7 shows the method for sizing an aortic graft using a functional sizer. FIG. 9 shows various embodiments of a functional sizer suitable for aortic valve repair.

The aortic valve 700 (FIG. 7A) comprises three semilunar valve cusps (or leaflets) 620 which coapt when the ventricles stop ejecting. The three leaflets 620 are hinged to an annulus GOO. Each third of the annulus forms a curved shape that is lowest at its midpoint and highest at the edges. The highest aspect of each leaflet 620 touches an adjacent leaflet 620. This point is called the commissure 730 and is attached to the wall of the aorta.

The part of the aorta to which the leaflets 620 attach bulges and is called the aortic root (item 710 in FIG. 7A). Each leaflet 620 attachment forms its own bulge and these are known as the sinuses of Valsalva. The coronary arteries come off the aorta at the root. Above the aortic root 710, the ascending aorta 720 tapers to a simple tubular structure. The point of tapering is the sino-tubular junction 715.

Different causes of aortic valve incompetence include:
1) a dilated annulus, so the leaflets do not close (similar to the mitral annulus dilatation—see FIG. 7B;
2) a dilated STJ, so the attachment points are moved outward—illustrated in ghost in FIG. 9N;
3) an aortic aneurysm—illustrated in ghost in FIG. 9O;
4) excess leaflet tissue that ‘flops’ backwards.

The aortic valve 700 can be repaired by ‘remodelling’ the aortic annulus, aortic root (see FIG. 9O) or the STJ (see FIG. 9N). The main cause of aortic valve leakage is an aortic aneurysm—i.e. the aorta is pathologically dilated. FIG. 9P is a side view of an aortic aneurysm repair in cross section showing the aortic aneurysm in ghost (grey dashed lines). To repair the aortic valve 700 in this situation the aortic wall is cut out leaving just the attachment of the valve. The ascending aorta 720 is replaced with an artificial tube graft 725—a supracoronary graft (see FIG. 9P). An aortic root graft 735 replaces the aortic root 710. The repair is shown superimposed over the aortic aneurysm in FIG. 9P to illustrate the effect of each respective graft. A functional sizer corresponding to a predetermined size of a supracoronary graft and/or an aortic graft is used to determine and test the appropriate size of each graft.

To size an aortic valve and corresponding prostheses for valve repair (e.g. a supracoronary tube graft for repair of STJ dilatation as illustrated in FIG. 7B), sutures 510 are placed in the annulus 600 (FIG. 7B). Sutures 740 are also positioned at the tops of the commissures 730. A ring-like or tube-like functional sizer (e.g. as illustrated in FIGS. 9A to 9P) can be used to model an annuloplasty ring or a tube graft, as desired.

A functional sizer 940 (see FIG. 9C) is selected and mounted to an applicator 160 (FIG. 7C). Alternatively, a functional sizer of the form shown in FIG. 9C but with a permanently fixed applicator (such as depicted in FIG. 11) is selected. The functional sizer (illustrated in FIG. 9C as embodiment 940 of FIG. 9C) is positioned above the annulus 600 (FIG. 7D) with the annulus sutures 510 and commissure sutures 740 underneath. The functional sizer 940 is then positioned against the annulus 600. Sutures 510, 740 are drawn up through the centre of the functional sizer 940 and placed under tension—indicated by arrows X (FIG. 7E), to secure the functional sizer 940 in position on the annulus 600. This is achieved by attaching the threads 510, 740 to a securing device of some sort—for example, a valve suture guide or ratcheted forceps that clamp the threads 510, 740 and is then placed on the patient's chest.

In this way, the aortic valve annulus 600 is reshaped or conforms to the shape of the functional sizer 940—the annulus 600 is no longer dilated and the leaflets 620 are drawn together, to close the valve 700.

The performance of the valve 700 is tested (FIG. 7F) while the functional sizer 940 is secured to the annulus (here, by placing the sutures in the annulus under tension). Saline is injected in the directions indicated by arrows Z. If there is no leakage, this indicates a good “fit” of the functional sizer 940 to the annulus 600 and that the valve 700 is closed (functioning correctly).

As the method of securing the functional sizer 940 in position is readily reversible, the functional sizer 940 can be removed without damage to the heart valve annulus. A tubular graft that corresponds in size to the functional sizer can then be fixed permanently, having previously, gauged the appropriate size using the functional sizer.

A similar method can be utilised with any of the embodiments 920 to 980 of FIGS. 9A to 9L for aortic valve annulus sizing. Embodiments 950 (FIG. 9D), 970 (FIGS. 9I) and 980 (FIG. 9L) secure the functional sizer in position, with tension using a self-clamping mechanism (see item 425 in FIG. 2C) that is placed around the sutures.

As the functional sizer in any embodiment is reversibly secured (that is, not stitched) to the heart valve annulus, in the event of a poor fit (e.g. continued leakage), the functional sizer can be released from position by releasing tension on the sutures and removing the functional sizer from its position on the annulus using an applicator. Alternatively, if the securing means is a self-clamping mechanism (e.g. item 425 in FIG. 2C), the functional sizer is released by opening the self-clamping mechanism. If the securing mechanism is a magnetised cap (e.g. item 520 of FIG. 2D), the functional sizer is released by removing the magnetised cap. A functional sizer 100 with a different internal diameter (circumference) can then be tried until a suitable fit is identified without damage to the suture threads or to the heart tissue.

The functional sizer is made of any sterilisable material (e.g. a suitable plastic, metal or composite material). This allows the functional sizer to be sterilised for reuse. This helps minimise waste and expense, and is an advantage over existing methods which require a permanently fixed but ill-fitting annuloplasty ring to be discarded if the surgeon decides to “re-size” the annuloplasty ring by replacing it.

As described above, the functional sizer is used to simulate the effect of an implantable device for heart repair or replacement surgery (e.g. an annuloplasty ring, a tubular graft, an artificial valve (valve replacement) by temporarily reshaping the heart valve to the shape of the functional sizer. The functional sizer temporarily reshapes:
(a) the annulus of any heart valve for valve replacement;
(b) the aorta or pulmonary artery at the top of the commissures for aortic or pulmonary valve repair;
(c) the mitral or tricuspid valve for repair.

In any embodiment, the ability to reshape the valve arises from securing the functional sizer in position in the heart. In embodiment 100 (FIGS. 1A and 3A), threads 510 previously sutured to the heart valve annulus are brought up and over the body 110 of the functional sizer, through the interior or centre of the ring-like shape (as shown in FIG. 3A). The suture threads 510 sit against the internal circumference of the functional sizer (see FIG. 3A). The functional sizer is then placed in position in the heart (e.g. against the valve annulus in valve repair surgery) and the sutures brought under tension. This will cause the annulus to conform to the same shape of the functional sizer.

Embodiment 100 is suitable for use for mitral or tricuspid valve repair. A similar method is used to secure in position the embodiments for aortic or pulmonary. valve repair, and the embodiments for valve replacement (e.g. see FIGS. 8C, 8D, 9E, 9H, 10A and 10B).

In this way, the functional sizer simulates the function of a correspondingly sized and shaped valve implantable device (e.g. annuloplasty ring, tube graft, or valve replacement). This allows the fit and performance of a correspondingly shaped and sized valve implantable device to be tested before permanently fixing a permanent implantable device.

In an embodiment, the functional sizer further includes securing means to assist in retaining the functional sizer in position more securely. The securing means are still reversible to enable the functional sizer to be secured in position temporarily and without damaging the sutures.

The securing means includes one or more of the following:
(a) preformed suture holes 210 on the body 110 of the embodiment 200 of FIG. 2A;
(b) preformed suture slots 310 on the body 110 of the embodiment 300 of FIG. 2B;
(c) a self-clamping, mechanism 425 including a resilient body 110 moveable between an open position and a closed position, and biased toward the closed position (e.g. the embodiments of FIGS. 2C, 9D, 9I-9M);
(d) a secondary ring-like body (magnetic cap 520) secured to the uppermost surface (the surface facing away from the heart annulus toward the surgeon) of the functional sizer (see FIGS. 2D to 2F, and 10C). The secondary ring is of substantially equivalent size (diameter) to the functional sizer and magnetised so that the two ring-like shapes (bodies) are held together closely and strongly. Pins 530 (FIGS. 2D, 10C) protrude from the inner surface of the magnetic cap 520 (the surface facing the functional sizer). The pins 530 mate with corresponding recesses 530 on the uppermost surface of the body 110 of the functional sizer to assist with positioning the magnetic cap 520 on the functional sizer. The magnetised cap 520 secures the sutures 510 in position on the functional sizer. The edges of the facing surfaces of the two ring-like shapes have rubber edging 540 for facilitating grip of the sutures 510.

In embodiments 200 (FIGS. 2A, 3B) and 300 (FIGS. 2B, 3C), suture threads 510 are fed through the securing means (e.g. the suture holes 210 of FIG. 2 or the suture slots 310 of FIG. 3) and held under tension using any suitable means such as ratcheted forceps. These embodiments are suitable for mitral or tricuspid valve repair. Similar securing means (preformed suture holes or slots) are also used in embodiments suitable for aortic or pulmonary valve repair (e.g. FIGS. 9B, 9F, 9G), or for valve replacement (e.g. FIGS. 10A and 10B).

In embodiments 200 (FIGS. 2A and 3B), 920 (FIGS. 9A, 9E), 940 (FIGS. 9C, 9H) and 1070 (FIG. 10A), sutures 510 are threaded or fed through suture holes 210 that go through the body 110 of the functional sizer (e.g. as shown in FIG. 3B), from lowermost surface (the surface positioned against the heart valve annulus) to uppermost surface (the surface facing away from the heart valve annulus, toward the surgeon). The surgeon places the sutures through the preformed holes 210 as they would for a permanently implantable device. The functional sizer 200 is then placed against the heart valve annulus with an applicator 160 (e.g. see FIGS. 6D, 7D) and the sutures placed under tension.

The suture holes 210 combined with placing the sutures under tension allows accurate placement and securing of the functional sizer on the underlying heart valve annulus.

In embodiments 300 (FIGS. 2B, 3C), 930 (FIG. 9B), 960 (FIG. 9G) and 1080 (FIG. 10B), the functional sizer includes large slots in the outer circumference of the body 110 that travel through the body 110 of the functional sizer (e.g. as shown in FIG. 3C), from lowermost surface (the surface positioned against the heart valve annulus) to uppermost surface (the surface facing the surgeon). Sutures 510 are threaded or slid through the suture slots 310 for accurate placement of the functional sizer on the heart valve annulus.

The surgeon places the functional sizer close to the annulus and inserts the sutures into the slots. The functional sizer is then applied to the heart with the sutures under tension. The suture slots 310 combined with placing the sutures under tension allows accurate placement and securing of the functional sizer on an underlying heart valve annulus.

In embodiments 400 (FIG. 2C), 950 (FIGS. 9D), 980 (FIG. 9L), the securing means is a self-clamping mechanism 425. The embodiment illustrated in FIG. 2C is suitable for mitral or tricuspid valve repair. An analogous self-clamping mechanism (e.g. a spring hinge to enable the functional sizer to be opened and closed) can be used on embodiments for aortic or pulmonary valve repair (see FIGS. 9D and 9I), tube grafts (FIGS. 9I and 9L) and artificial valve replacements (e.g. FIGS. 10A to 10C).

The ring-like or tube-like body of the embodiments 400, 950 and 980 with a self-clamping mechanism is moveable between:
(a) a closed position (as shown in FIGS. 4A, 9J, 9L), in which the body takes the shape of a closed loop; and
(b) an open position (as shown in FIGS. 4B, 9K, 9M), in which the ring-like shape is an open or incomplete loop.

This enables the functional sizer to be opened for positioning on a heart valve annulus, and then closed to secure the functional sizer in position.

The ring-like body 110 of embodiments 400, 950, 980 is made of opposable parts 410 (FIG. 2C, 9K, 9M). The opposable parts 410 are:
(a) moveable apart (in the direction shown by arrow A in FIG. 2C) to move the body toward an open position; and
(b) moveable together (in the direction shown by arrows B) to return the body toward a closed position.

The functional sizer 400, 950, 980 includes a biasing means to bias the opposable parts 410 together (and thereby biasing the body to a closed position). The biasing means includes one or more of the following:
(a) a spring-loaded hinge to bias the opposable parts together (shown schematically as a spring hinge 420 in FIG. 2C);
(b) a locking mechanism to lock the opposable parts together (not shown);
(c) a resilient material to bias the opposable parts together (not shown).

In an arrangement, the functional sizer is made of any suitable material and hinged (without a spring) so that the opposable parts 410 are moveable apart and together, about the hinge. To retain the functional sizer in a closed position, a locking mechanism is provided on the opposable parts 410 such that once opposed the opposable parts 410 click or lock together securely to lock the functional sizer in a closed position. The locking mechanism can be any suitable locking mechanism such as an interference fit, press fit, corresponding catches on opposing parts, or a latch mechanism.

In another arrangement, the functional sizer 400, 950, 980 are made from a resilient material that biases the opposable parts together. The body 110 is moved toward the open position by applying pressure against the bias. Once the pressure is released, the body 110 returns to a closed position.

In the body 110 of embodiment 400, 950, 980 shown in FIG. 2C, the biasing means is shown as a spring hinge 420 (shown in ghost) positioned diametrically opposite the opposable parts 410. The spring hinge 420 biases the body 110 toward a closed position.

Embodiment 400, 950, 980 includes a pair of flanges 430 (e.g. see FIG. 2C). The flanges 430 extend tangentially from the body diametrically opposite the opposable parts 410. Each flange 430 is at an acute angle (ideally 90 degrees, but any angle between around 30 to 90 degrees should also work) to the other flange of the same pair. Pressing the flanges together (in direction shown by arrows C) moves the opposable parts 410 apart (by approximately 90 degrees) thereby moving the body toward an open position. The flanges 430 are lockable together (with a clamp or similar) so that the ring-like body is lockable in an open position.

In use, the opposable parts 410 of the body 110 will be positioned on the heart valve annulus so that the when the functional sizer 400, 950, 980 is moved to an open position (e.g. see FIGS. 4B, 9K and 9M), the opening 900 is on the same side as the dominant hand of the surgeon. This allows surgeons to use their dominant hand to position suture threads 510 inside the open ring-like shape whilst the threads are still under tension.

The ring-like shape can be locked in the open position using a locking mechanism to lock the flanges 430 together. An example of a suitable locking mechanism is a readily available ratcheted surgical clamp (or a specially made one) such as the surgical clamp 910 shown in FIGS. 2C, 4B, 9K and 9M, which can be applied to the flanges 430 when opposed to lock flanges together and thereby to lock the ring-like body 110 of the functional sizer in an open position.

The open ring-like body 110 is placed around the suture threads 510 at a level well above the valve annulus. The sutures 510 are placed under tension and the locking mechanism released (e.g. releasing the surgical clamp 910) to free the flanges 430 (see FIGS. 4B, 9K and 9M). Removing the clamp 910 allows the opposable parts 410 of the ring-like body (refer FIG. 4) to move to a closed position (arrows B in FIG. 2C) and form the closed loop shape of an annuloplasty ring.

By placing the suture threads 510 under tension, force is applied on the functional sizer 400 to further secure the functional sizer 100 in position on the heart valve annulus. As with other embodiments, securing the functional sizer in position causes the annulus to conform to the same shape as the functional sizer (which reflects the size and shape of a permanent annuloplasty ring). In this way, the secured functional sizer simulates the effect of an annuloplasty ring of a corresponding size.

The functional sizer 800 embodies a partial ring with flanges 805 directed towards the inner diameter. This allows for annular sutures 510 to be placed securely within the functional sizer 800 which are prevented from sliding out of the partial ring 800 by the flanges 805. This embodiment 800 represents and simulates any partial annuloplasty ring/band for mitral/triscupid valve repair. Similarly this partial ring 800 could also consist of a slotted functional sizer 300 or hinged mechanism as in functional sizer 400.

In an embodiment, the functional sizer 810 embodies a ring with a three dimensional structure that is not planar thereby simulating permanent annuloplasty rings that are not planar. The heart valve annulus would conform to this three dimensional shape by placing the sutures 510 through the centre of the ring and putting them under tension whilst the functional sizer 810 is held against the annulus 600. Similarly this three dimensional structure 810 could also consist of a slotted functional sizer 300 or hinged mechanism as in functional sizer 400.

In any embodiment, the shape of the secured functional sizer allows testing using conventional means for assessing fit and functionality (prevention of leakage) during valve repair or replacement surgery. A conventional testing method to test the function of the leaflets once the annulus is repaired involves injecting saline into the ventricle.

An advantage of the preferred embodiments over conventional methods is that the functional sizer allows testing the performance of the leaflets before permanently fixing an annuloplasty ring. Testing can be undertaken once the functional sizer is secured in place, rather than after the actual repair, once an annuloplasty ring is permanently fixed in.

The functional sizer is also useful as a valve replacement template for temporarily securing a valve replacement in position in the heart—see embodiments 1070, 1080 and 1090 in FIGS. 10A, 10B and 10C. Currently a valve replacement template is used but is not fixed and has to be held in by the surgeon.

The advantage of the functional sizer 1070, 1080, and 1090 is that a surgeon can temporarily fix (secure) the functional sizer in position (similarly as described above for temporarily securing an annuloplasty ring or tube graft). Embodiments 1070, 1080 and 1090 suitable for heart valve replacement (e.g. shown in FIGS. 10A to 10C) can be fixed in position for testing using suture holes 210, suture slots 310 or a magnetic cap 520 as the securing means.

In an embodiment, the functional sizer is capable of functioning as an implantable device suitable for heart valve repair and/or replacement. This is achieved by permanently fixing the functional sizer using a permanent securing means such as by tying the sutures, or by retaining one of the reversible securing means described above in a secured state. For example, a functional sizer that includes a magnetic cap could be used to secure the functional sizer in position.

Fixing the functional sizer 1070, 1080, 1090 in position allows the impact on the other valves to be assessed. This is particularly advantageous, for instance, during a double valve replacement, because having two valve replacements in situ can cause distortion of the heart and ultimately reduce the available space for the replacement. Currently, surgeons ‘down-size’ the valves when doing a double valve replacement. A functional sizer removes the guesswork as to whether positioning two valves close together will fit. This is because one or more functional sizers can be secured in position and tested for fit and performance before fixing one or more permanent valve replacements of respective corresponding sizes.

A further advantage of the preferred embodiments is that they enable more accurate repairs which involve implantation of new chords (neo-chords—item 630 in FIG. 6A) for ruptured or redundant chords. Currently surgeons use a variety of means to measure the length of the new chords including visual, in-situ measurement, or radiographic measurement. However, all of these measurements are somewhat arbitrary as the required length of the neochords is governed by the final annulus shape and size which is altered by the annuloplasty ring.

Surgeons usually measure and implant the chords and then attach the annuloplasty ring as the chords are difficult to implant and adjust with the annuloplasty ring in-situ. Often the length of the neochords is found to be inaccurate after the ring is implanted. The preferred embodiments allow for a remodelling of the annulus (through securing of a functional sizer to the annulus) whilst the chords are being measured/implanted. Although developed as a device and method for temporarily reshaping the annulus, because the functional sizer simulates the function of an annuloplasty ring, it can also be left in place permanently if so desired.

The invention provides a device and method for assessing fit and testing function of an implantable heart valve repair or replacement device during heart valve repair or replacement without needing to first stitch in the implant. The device simulates a functional heart valve repair implant or replacement prosthesis (e.g. annuloplasty ring, graft or valve replacement) of a corresponding size providing an opportunity for testing before permanent fixation to the heart valve.

An advantage of the preferred embodiments is that they allow a surgeon to assess fit and test function of a heart valve before permanently fixing an implantable heart valve device, thereby avoiding the risk of ill-fit, or the need to replace a permanently fixed repair implant or replacement prosthesis (with attendant risks of damage to stitches and risk to the patient).

A further advantage of the preferred embodiments is that they provide a heart valve repair or replacement implantable device (annuloplasty ring, graft or valve replacement) that can be reversibly secured to a heart valve annulus without damaging the sutures or risk to the patent in releasing the implantable device.

The invention provides a device and method for sizing a heart annulus for heart valve repair or replacement surgery. However, it will be appreciated that the invention is not restricted to these particular fields of use and that it is not limited to particular embodiments or applications described herein.

Comprises/comprising when used in this specification is taken to specify the presence of stated features, integers, steps or components but does not preclude the presence or addition of one or more other features, integers, steps, components or groups thereof.” Thus, unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to.

1. A functional sizer for sizing an implantable heart valve device for heart valve repair or replacement, the functional sizer comprising a ring-like body with a central hole, the ring-like body being of a sterilisable material,
wherein the ring-like body is one of the following two-dimensional shapes:
(a) a closed loop;
(b) a penannular shape, and
wherein the ring-like body substantially corresponds in inner circumference to a ring of suture threads positioned around a heart valve annulus for securing a heart valve implantable device of a predetermined size, each suture thread of the ring of suture threads having a free end
wherein the functional sizer is adapted to be reversibly secured in position against the heart valve annulus, the reversibly secured functional sizer simulating an effect of a heart valve implantable device of a substantially corresponding size and form, thereby facilitating assessment of fit and function of the heart valve implantable device.

2. The functional sizer of claim 1, wherein the ring-like body is one of the following three-dimensional forms:
(a) substantially planar;
(b) tube-like;
(c) bulbous;
(d) a three-dimensional bent ring-like form
(e) another three-dimensional ring-like form.

3. The functional sizer of claim 1 wherein the ring-like body is adapted to be reversibly secured in position against the heart valve annulus by drawing the free ends of said suture threads up through the ring-like body from underneath to over the ring-like body, then placing the one or more suture threads under tension, such that when reversibly secured in position against the heart valve annulus by placing the one or more suture threads under tension, the functional sizer simulates an effect of a heart valve implantable device of a substantially corresponding size and form, thereby facilitating assessment of fit and function of the heart valve implantable device.

4. The functional sizer of claim 1 further including a securing means for reversibly securing the functional sizer in position against a heart valve annulus.

5. The functional sizer of claim 4 wherein the securing means includes one of the following:
(a) a self-clamping mechanism;
(b) a magnetised cap;
(c) one or more suture holes passing through the ring-like body from lowermost surface to uppermost surface for threading said free ends of said suture threads through said ring-like body, the lowermost surface being the surface of the ring-like body positioned against the heart valve annulus when in use and the uppermost surface being the surface of the ring-like body facing away from the heart valve annulus;
(d) one or more suture slots passing through the ring-like body from the lowermost surface to the uppermost surface for threading said free ends of said suture threads through said ring-like body.

6. The functional sizer of claim 5 wherein the self-clamping mechanism includes:
(a) a ring-like body comprising opposable parts, wherein the ring-like body is moveable between an open position and a closed position and wherein the opposable parts are opposed when the body is in a closed position;
(b) a biasing means to bias the opposable parts together such that the functional sizer is biased in a closed position with the opposable parts biased together, and moveable to an open position by moving the opposable parts of the ring-like body apart.

7. The functional sizer of claim 6 wherein the ring-like body is hinged such that the opposable parts are moveable apart and together, about the hinge.

8. The functional sizer of claim 6 wherein the biasing means is one of the following:
(a) a spring-loaded hinge to bias the opposable parts together;
(b) a locking mechanism to lock the opposable parts together;
(c) a resilient material to bias the opposable parts together.

9. The functional sizer of claim 8 wherein the functional sizer further includes a pair of flanges extending tangentially from the body diametrically opposite the opposable parts, wherein each flange of a pair is at an acute angle to the other flange of the same pair such that pressing the flanges of a pair together moves the opposable parts apart.

10. The functional sizer of claim 5 wherein the magnetised cap includes a secondary ring-like body of a substantially equivalent size and two-dimensional shape to the ring-like body of the functional sizer, and
wherein the secondary ring-like body is magnetised, such that when the magnetised cap is placed on the ring-like body of the functional sizer, the two ring-like bodies are held together by magnetic force,
wherein any suture threads drawn up and over the ring-like body of the functional sizer are held in position on the ring-like body by the magnetised cap and releasable by removing the magnetised cap.

11. The functional sizer of claim 10 wherein the functional sizer further includes rubber edging on facing surfaces of the two ring-like bodies to facilitate grip of the suture threads between the two ring-like bodies.

12. The functional sizer of claim 1 wherein the ring-like body further includes a mounting means for mounting the ring-like body to an applicator to enable the functional sizer to be handled by handling the applicator.

13. The functional sizer of claim 12, wherein the mounting means is one of the following: (a) permanent;
(b) reversible.

14. The functional sizer of claim 12 wherein the applicator comprises a shaft extending between the ring-like body and a handle means.

15. The functional sizer of claim 14 wherein the shaft is made of malleable material.

16. The functional sizer of claim 1 wherein the functional sizer is further adapted to be permanently secured in position on a heart annulus by one of the following:
(a) stitching to the heart annulus;
(b) retaining the self-clamping mechanism in a closed position;
(c) retaining the magnetised cap on the ring-like body of the functional sizer.

17. A functional sizer for sizing an implantable heart valve device for heart valve repair or replacement, the functional sizer comprising a ring-like body with a central hole, the ring-like body being of a sterilisable material,
wherein the ring-like body is one of the following two-dimensional shapes:
(a) a closed loop;
(b) a penannular shape, and
wherein the ring-like body substantially corresponds in inner circumference to a ring of suture threads positioned around a heart valve annulus for securing a heart valve implantable device of a predetermined size, each suture thread of the ring of suture threads having a free end
wherein the functional sizer is adapted to be reversibly secured in position against the heart valve annulus, the reversibly secured functional sizer simulating an effect of a heart valve implantable device of a substantially corresponding size and form, thereby facilitating assessment of fit and function of the heart valve implantable device, and wherein said functional sizer is further adapted to be permanently secured in position on a heart annulus by one of the following:
(a) stitching to the heart annulus;
(b) retaining the self-clamping mechanism in a closed position;
(c) retaining the magnetised cap on the ring-like body of the functional sizer.

18. A method of sizing a heart valve implantable device including the steps of:
(a) positioning a functional sizer in a heart, wherein the functional sizer is a ring-like body of sterilisable material of an approximately corresponding size to a heart valve implantable device of a predetermined size;
(b) reversibly securing the functional sizer in position.

19. The method of claim 18 wherein reversibly securing the functional sizer includes the steps of:
(a) drawing free ends of a ring of suture threads up through the ring-like body from underneath to over the ring-like body;
(b) placing the suture threads under tension after drawing the free ends up through and over the ring-like body.

20. A method of sizing a heart valve implantable device including the steps of: (a) positioning a functional sizer against a heart valve annulus,
wherein the functional sizer comprises a ring-like body with a central hole, the ring-like body being of a sterilisable material, wherein the ring-like body is one of the following two-dimensional shapes:
i. a closed loop;
ii. a penannular shape, and
wherein the ring-like body substantially corresponds in inner circumference to a ring of suture threads positioned around the heart valve annulus for securing a heart valve implantable device of a predetermined size, each suture thread of the ring of suture threads having a free end;
(b) drawing the free ends of said suture threads up through the ring-like body from underneath to over the ring-like body;
(c) placing the one or more suture threads under tension after drawing the free ends up through and over the ring-like body such that when reversibly secured in position against the heart valve annulus by placing the one or more suture threads under tension, the functional sizer simulates an effect of a heart valve implantable device of a substantially corresponding size, shape and form, thereby facilitating assessment of fit and function of the heart valve implantable device.

21. The method of claim 20 including the further step of testing performance of a heart valve while the functional sizer is reversibly secured in position against the heart valve annulus.

22. The method of claim 21 wherein testing performance of a heart valve includes a substep of injecting water into the heart valve while the functional sizer is reversibly secured in position.

23. The method of claim 20 including a further step of releasing the functional sizer from its position on the heart valve annulus.

24. The method of claim 20 including a further step of testing one or more functional sizers, each with a different internal circumference and each reversibly secured to the heart valve annulus for testing, until a suitable fit is identified.

25. The method of claim 20 including a further step of permanently securing a functional sizer in position on a heart valve annulus after testing performance of the heart valve while the functional sizer is reversibly secured in position, wherein the step of permanently securing includes one of the following steps:
(a) stitching the functional sizer to the heart annulus;
(b) retaining the self-clamping mechanism of the functional sizer in a closed position;
(c) retaining the magnetised cap on the ring-like body of the functional sizer.
